(19) United States
(12) Patent Application Publication
(10) Pub. No.: US 2011/0319298 A1
(43) Pub. Date: Dec. 29, 2011

(54) Differential detection of single nucleotide polymorphisms

(76) Inventors: Steven A. Benner, Gsinesville, FL (US); Shuichi Hoshika, Gainesville, FL (US); Nicole Leal, Gainesville, FL (US)

(21) Appl. No.: 12/311,982

(22) PCT Filed: Apr. 21, 2009
(86) PCT No.: PCT/US2009/002463

§ 371 Date: Apr. 21, 2009

(51) Int. Cl. C40B 50/06; C07H 21/00

(52) U.S. Cl. 506/26; 536/23.1

(57) ABSTRACT

This patent application claims processes and compositions of matter that enable the discovery of single nucleotide polymorphisms (SNPs) that distinguish the genomes of two individual organisms in the same species, as well as that distinguish the paternal and maternal genetic inheritance of a single individual, as well as distinguish the genomes of cells in special tissues (e.g. cancer tissues) within an individual from the genomes of the standard cells in the same individuals, as well as the SNPs that are discovered using these processes and compositions. Two steps are essential to the invention disclosed in this application. The first step provides four sets of primers, which are designated “T-extendable”, “A-extendable”, “C-extendable”, and “G-extendable”. These primers, when targeted against a reference genome as a template, add (respectively) T, A, C, and G to their 3′-ends in a template-directed primer extension reaction. The second step presents these four primer sets, separately, to a sample of the target genome DNA under conditions where they bind to their complementary segments within the target DNA. Once bound, members of each primer set serve as primers for a template-directed primer extension reaction using the target genome as the template. If the template from the target genome presents the same templating nucleotide for the first nucleotide added in the extension reaction as the reference genome, then the T-extendable, A-extendable, C-extendable, and G-extendable primers will be extended (respectively) by T, A, C, and G. If, however, the template from the target genome presents a nucleotide different from the reference genome, then the T-extendable, A-extendable, C-extendable, and G-extendable primers will be extended (respectively) by not T, not A, not C, and not G (referred to here as “3N” or “3”, to indicate the other three nucleotides, where which of the other three is understood by context). In these cases, the primers have discovered a SNP, a difference between the target and reference genomes. Then, the T-extendable, A-extendable, C-extendable, and G-extendable primers that add (respectively) not-T, not-A, not-C, and not-G are separated or made otherwise physically distinct (through, for example, the use of irreversible terminators, such as 2′,3′-dideoxynucleosides) from those that added T, A, C, and G (respectively). Those that added T, A, C, and G (respectively) did not discover a SNP, and are discarded. The primers that added “not-T”, “not-A”, “not-C”, and “not-G” discovered a SNP, and presented in a mixture enriched (relative to those primers that did not discover a SNP) in a useful deliverable. Following these steps, the SNPs discoveries are realized by sequencing the extracted species. The information obtained from this sequencing allows the identification of the locus of the SNP in the in silico genome.

### FIELD OF THE INVENTION

This invention relates generally to processes and compositions for analyzing DNA sequences from organisms, and more particularly to methods and compositions for discovering single nucleotide variations, or “polymorphisms”, sites in a sequence of DNA that hold a nucleotide that is different from the nucleotide in the analogous site in the analogous sequence, both within a diploid individual and between two individuals in the same species. This invention also claims those SNPs discovered using the processes and compositions of the instant invention.

### BACKGROUND

Genetic variation distinguishing the genomes of individuals within a species of organisms is a major, if not the major, determinant of the differential responses of those individuals to different environments, their differential susceptibility to disease, and (in medicine, human or animal) their differential response to various therapeutic regimens. Accordingly, discovering genetic differences (such as “single nucleotide polymorphisms”, or SNPs) between different individuals, between tissues within an individual (such as those that arise in cancer tissues), or even between analogous sites in chromosomes in a diploid individual (which shows the differences in the genetic material received from the two parents) is a major goal of research in many laboratories. SNP discovery and detection is therefore emerging as a major theme in research on many species (including bacteria, animals, fungi, and plants), and in human and animal medicine. Direct evidence for the utility of any tools that discover or detect variation of this type is the number of National Institutes of Health (NIH) opportunities for funding research to develop such tools (for example RFA-HL-08-004).

“SNP discovery” is fundamentally a different problem from “SNP detection”. The second presumes that one already knows the variant sequence that one wishes to detect. Knowing what one wants to find makes finding it easier, of course, and many tools are available for identifying known single nucleotide polymorphisms (SNPs) in a sample of DNA (from a patient, for example) [Sjo08] [Kim08]. In contrast, very few tools exist for the high-throughout discovery of unknown genetic variations.

Many approaches in the art to discover SNPs simply do standard DNA sequencing on the genomes (or parts of genomes) of many individuals. We call these “brute force” approaches”. For example, the combined work of the SNP Consortium [Sac01] and other public projects has discovered ˜10 million SNPs in various human genomes just by sequencing. The work continues in an NIH program to re-sequence many different cancer tissues, hoping that variation between cell types (cancerous, non-cancerous) that is significant to the cancer disease is not lost amid irrelevant variation arising from the “mutator phenotype” of cancer cells.

A non-brute force approach for discovering single nucleotide differences that distinguish a target genome from a reference genome is the cell-based approach described by Faham et al. [Fah01][Fah05] (the terms “target” and “reference” will be used throughout this disclosure; the distinction is theoretically arbitrary, but is needed in the context of descriptions of specific architectures). This approach exploits the mismatch repair system in vivo in E. coli. Mismatch repair detection (MRD) was used [Fak04] in the search for SNPs that separate cancer cell genomes from the genomes in their untransformed counterparts [Pet07]. Here, the technique permitted a search limited to 10.3 Mb (ca. 0.3%) of the tumor genome, or ca. 8.5 Mb of protein coding sequence. Approximately 90% of the amplicons screened showed a perfect match to the reference genome sequence. An additional 8.7% of the amplicons had variations that distinguished them from the corresponding matched normal samples, suggesting these were likely germ line variations. These were also removed from subsequent analysis. The remaining 0.3% of amplicons were sequenced to discover 54 putative somatic mutations.

Brute force approaches for SNP discovery in various species are assisted today by the fact that often, a whole genome sequence for an individual of that species has been determined and is recorded in a computer database (an in silico genome). For humans, this is the case as well. In this case, we speak of “re-sequencing”, rather than “de novo sequencing”. Brute force re-sequencing is less expensive than de novo sequencing because without an in silico sequence, short fragments of DNA sequence determined in the sequencing experiments must be assembled into a closed chromosome using only information from other short fragments. In resequencing, fragment assembly is guided by the in silico genome. This is simpler, in the same was as assembling a jigsaw puzzle is simpler when the pieces can be laid on top of a picture of the puzzle.

### SUMMARY OF THE INVENTION

The instant invention discovers single nucleotide polymorphisms (SNPs), different nucleotides present in analogous sites in two sequences, by a process involving these essential steps. The first step provides four sets of primers, which are designated “T-extendable”, “A-extendable”, “C-extendable”, and “G-extendable”. These primers, when targeted against a reference genome as a template, add (respectively) T, A, C, and G to their 3′-ends in a template-directed primer extension reaction.

The second step presents these four primer sets, separately, to a sample of the target genome DNA. Members of each set are contacted to the target genome DNA in buffer appropriate for them to bind to their complementary segments within the target DNA. Once bound, members of the primer set serve as primers for a template-directed primer extension reaction using the target genome as the template. If the template from the target genome presents the same templating nucleotide for the first nucleotide added in the extension reaction as the reference genome, then the T-extendable, A-extendable, C-extendable, and G-extendable primers will be extended (respectively) by T, A, C, and G. If, however, the template from the target genome presents a nucleotide different from the reference genome, then the T-extendable, A-extendable, C-extendable, and G-extendable primers will be extended (respectively) by not T, not A, not C, and not G (referred to here as “3N” or “3”, to indicate the other three nucleotides, where which of the other three is understood by context). In these cases, the primers have discovered a SNP, a difference between the target and reference genomes.

Then, the T-extendable, A-extendable, C-extendable, and G-extendable primers that add (respectively) not-T, not-A, not-C, and not-G are separated or made otherwise physically distinct (through, for example, the use of irreversible terminators, such as 2′,3′-dideoxynucleosides) from those that added T, A, C, and G (respectively). Those that added T, A, C, and G (respectively) did not discover a SNP, and are discarded. The primers that added “not-T”, “not-A”, “not-C”, and “not-G” discovered a SNP, and presented in a mixture enriched (relative to those primers that did not discover a SNP) in a useful deliverable.

The discoveries of the SNPs may be realized by sequencing the primers immediately preceding the added nucleotide. The information obtained from this sequencing allows us to identify the locus of the SNP in the in silico genome.

This specification teaches the distinction between the invention and the architecture used to implement the invention. The architecture used to execute this process must ensure that the length of the primer is sufficient to carry enough information to identify the locus of the SNP in the corresponding in silico genome, at least in a useful number of cases to a useful degree of uniqueness. This length depends on the nature of the genome being probed. More information is required to locate a SNP in a larger genome than in a smaller one. Further, depending on the nature of the genome being probed, special arrangements are made to handle heterozygosity (in diploid genomes) and the repetitive “low-information content” nature of 90% of the human genome, reference and target.

Many architectures can be used to implement the instant invention. They differ (for example) in the way in which the four primer sets are provided, the way in which the N-extendable primers (where N is used to designate T, A, C, or G) that were extended with not-N are recovered, how the recovered sequences are extended, how specific challenges presented by a specific genomes are solved, and the extent to which an architecture trades coverage (the fraction of SNPs discovered) and cost. Various of these are discussed in the Detailed Description below, and exemplified in the Examples.

The teachings of this disclosure are inventive in multiple ways. First, they are inventive in the processes that they disclose that use physical DNA from a genome to generate four different primer sets. This may be considered to be inventively distinct (and prompt a restriction/election requirement) because such primer sets may be used for purposes other than to identify SNPs. Also inventive are the processes disclosed that exploit the primer sets; these might be considered to be inventively distinct (and prompt a restriction/election requirement) because of their other applications. Also inventive are the processes that deplete primers that prime against repeats from a downstream deliverable. Another of the inventive teachings of this disclosure is that determining the heterozygosity of a diploid individual provides a substantial sampling of the difference between the genome of the individual and the average genome of a population.

The most important invention, however, are the SNPs that are derived from the combination of all of these. Therefore, the claims that are presented in the instant invention require the inventive steps implemented by one of the architectures disclosed here. This invention cannot be subjected to a restriction/election requirement, because only the combination of steps will deliver this particular useful outcome, a collection of discovered SNPs within a genome.

### DEFINITIONS

454: The DNA sequencer that uses a SuCRT strategy based on pyrophosphate sequencing, developed by a Connecticut-based based developer of a sequencing using cyclic reversible termination (SuCRT) architecture.
5′-Bishomologated aldehyde DNA: A DNA molecule that, at its 5′-end, has the 5′-OH group replaced by a —CH₂CH₂CHO unit.
5′-Homologated aldehyde DNA: A DNA molecule that, at its 5′-end, has the 5′-OH group replaced by a CH₂CHO unit.
AEGIS: Artificially Expanded Genetic Information Systems, a kind of DNA that forms Watson-Crick pairs with DNA containing complementary AEGIS components, but not with natural DNA [Ben04].
Analogous segments: In the comparison of two genomes, we may speak of homologous segments and analogous segments. Homology is a theoretical term, and refers two segments in two genomes in two organisms that are related by common ancestry. Analogous is an operational term, and refers to two sequences that are largely identical over a significant length.
Architectures have, which have procedures and protocols.
DNA fragment: The physical piece of DNA, generally duplex.
DNA fragmentation: Breaking of the physical DNA into pieces. This is done, inter alia, by restriction digestion, sonication, or focused disruption using the Covaris instrument, all well known in the art, most preferably using the Covaris instrument when fragments 50-200 nucleotides are desired.
DNA Segment: The representation of a physical piece of DNA, which may be on paper or in a computer.
Exemplars: The number of copies of the analogous DNA segment in a mixture.
Explicit chemical synthesis: This refers to phosphoramidite synthesis of specific DNA sequences, under control of software, for example, and is distinct from the synthesis of sequences as part of library synthesis (e.g., split and pool, or through the addition of phosphoramidite mixtures).
homologous segments: In the comparison of two genomes, we may speak of homologous segments and analogous segments. Homology is a theoretical term, and refers two segments in two genomes in two organisms that are related by common ancestry. Analogous is an operational term, and refers to two sequences that are largely identical over a significant length.
HONO: Nitrous acid.
IBS: Intelligent BioSystems, a Waltham-based developer of a sequencing using cyclic reversible termination (SuCRT) architecture.
In silico genome: The computerized genome, from the public database.
Ligation-cleavage protocol: A way of generating N-extendable primers from polished duplex ends. Here, the blunt ended duplex fragments are ligated to the short duplex units that, upon restriction endonuclease treatment, generate the four primer sets, with the uncleaved species being terminated with a 2′,3′-dideoxynucleotide.
Locus: location, not placement.
—ONH₂: A capturable, reversible terminator, an alkyloxylamine.
Overhang: When reference is made to a 5′- or 3′-end, a single stranded extension preceding or following (respectively) a duplex region.
PEG: Polyethylene glycol.
Physical DNA: This refers to tactics where the DNA or RNA from a reference or target genome directly provides the material for the primer without an intervening in silico analysis, or the chemical synthesis of DNA. The physical DNA can be used directly. Alternatively, the physical DNA can be amplified by growth of the host organism, cloning followed by growth of the clones, or PCR amplification outside of a living cell.

### PMMA: Poly(methylmethacrylate).

Polishing: This refers to a process of rendering the DNA fragments blunt ended, either by removal of overhangs with nuclease digestion (e.g., with mung bean nuclease or Exo T, sold by New England Biolabs) of the single stranded overhangs and/or underhangs, or by polymerase filling in of 3′-underhangs by treatment with DNA polymerase and 2′-deoxynucleoside triphosphates (a fill in protocol). It is understood that failure to polish the ends of all duplex fragments need not be problematic in a stochastic process.
Polymerase: Includes DNA polymerases and reverse transcriptases.
POSaM: A piezoelectric ink jet instrument developed by Lausted et al. to synthesize two dimensional arrays of DNA sequences.
Process: In this disclosure, the process composed of two steps for delivering a collection of DNA fragments enriched in those containing, or adjacent to, sites that hold a nucleotide difference between a target and reference genome.
RNase: Ribonuclease; RNase A refers to one of various pancreatic RNases that are available commercially.
SAMS: Self-Avoiding Molecular Recognition System, a kind of DNA that forms Watson-Crick pairs with natural DNA, but not other SAMRS DNA.
SdS: Sequencing during synthesis, a strategy for parallel sequencing in various architectures, but where the sequence is determined as a primer is extended on an unknown template.
SNAP2: An architecture where two short fragments are assembled via a dynamic bond on a template under conditions of dynamic equilibrium; these fragments prime synthesis when the bond is formed.
SNP: Single nucleotide polymorphism.
Steps: In the instant invention, the first step in the Process is the procedure is the generation of four sets of primer sets, the G-extendable, A-extendable, C-extendable, and T-extendable primer sets. The second step is the use of these primer sets to discover fragments of DNA containing or adjacent to sites holding sequence variation. The third step recovers those fragments, delivering them as part of a mixture where they are enriched with respect to fragments that do not contain (or are not adjacent to) sites holding sequence variation.
SuCRT: Sequencing using cyclic reversible termination.
Reference genome: The genome that provides the “standard” sequence.
Target genome: One of a number of well-phenotyped genomes of interest to the NHLBI.
Underhang: When reference is made to a 5′- or 3′-end, this indicates that this end is preceded or followed (respectively) by a single stranded region on the complementary DNA.

### DETAILED DESCRIPTION OF THE INVENTION

Some general protocols are used in various architectures that implement the process of the instant invention.

For example, many architectures require fragmentation of a sample of duplex DNA. This can be done by simple sonication, to give duplex fragments between 1000 and 10000 nucleotide pairs in length. This will generate a fragment with an end at any particular site with a probability of one in 1000 to one in 10000. This is suitable, for example, when primers are being generated by exonuclease III digestion.

When shorter fragments are desired, for example, to get primer sets that can be used with immobilized templates, or templates that can be used with immobilized primers, or to have a higher chance of having a primer end at any particular site, focused fragmentation tools (such as those provided by an instrument sold by Covaris, Inc. (14 Gill Street, Unit H Woburn, Mass. 01801-1721) are used. These can generate, with relatively narrow length distributions, duplex fragments as short as 50 nucleotide pairs or as long as 1000 nucleotide pairs. The shortest fragments are as short as needed by the instant invention.

For some of the architectures that implement the process of the instant invention, the ends of the fragments need to be “polished”, that is, rendered to be blunt end. This is achieved either by removal of overhangs with nuclease digestion (e.g., with mung bean nuclease or Exo T, sold by New England Biolabs) of the single stranded overhangs and/or underhangs, or by polymerase filling in of 3′-underhangs by treatment with DNA polymerase and 2′-deoxynucleoside triphosphates (a fill in protocol). The second is generally preferred as the second protocol. It is understood that failure to polish the ends of all duplex fragments need not be problematic in a stochastic process.

In various architectures that implement the process of the instant invention, control over downstream processing involves control over ligation. This is done in several of the examples by control over 5′-phosphorylation.

In various architectures, different polymerases are needed having different properties. These are summarized in the Table below.

TABLE
Summary of Polymerase Activities

| | 2′,3′-dideoxy-nucleoside | 2′,3′-dideoxy-3′-amino | 2′-deoxy-3′-O-NH₂ nucleoside | 2′-deoxyribo-nucleoside | ribonucleoside |
|---|---|---|---|---|---|
| | triphosphate (irreversible | triphosphate (irreversible | triphosphate (reversible | triphosphate (extendable, | triphosphate (condi- |
| | terminator, not capturable, | terminator, capturable, | terminator, capturable, | not capturable, | tionally extendable, |
| 3′-terminus | not removable) | not removable) | not removable) | not removable) | capturable, removable) |
| ribo- | | | Taq475 | many | T7 RNA pol |
| nucleotide | | | | polymerases | (further |
| (permits | | | | (NEBL) | extension) |
| removal of | | | | | KlenE710A |
| following | | | | | (termination) |
| nucleotide) | | | | | PatelTaq |
| | | | | | (further |
| | | | | | extension) |
| 2′-deoxyribo- | TR-Taq | TR-Taq | Taq475 | all DNA | T7 RNA pol |
| nucleotide | Taq475 | | | polymerases | (further |
| | | | | (further | extension) |
| | | | | extension) | KlenE710A |
| | | | | | (termination) |

Taq475. E517G, K537I, L613A variant of Taq DNA polymerase
TR-Taq. A variant of Taq developed by Tabor and Richardson
KlenE710A. A variant of Klenow fragment of DNA polymerase [Ast98]
PatelTaq A variant of Taq developed by Patel and Loeb [Pat00]

### 1. Step 1. Providing the Sets of Primer Sequences

### 1.1 Direct Synthesis of Primer Sets of DNA

The most direct way to generate the four sets of primer sequences is by direct chemical synthesis. Here, the in silico genome is used as the reference genome. Subsequences are designed of length n (where n is chosen to allow a useful number of the primers to find unique templates in the target genome) by making reference to the in silico genome, and prepared by direct synthesis. These may be pooled, as it is known which n-mers are, when primed on the target genome template, are T-extendable, which are A-extendable, which are C-extendable, and which are G-extendable.

The advantage of direct synthesis is that the primers are obtained directly, without the need for priming reactions, cut backs, or other steps used in other architectures to prepare the primer sets are needed. Direct synthesis is, however, limited by the number of sequences that can be deliberately synthesized, which in turn limits the number of sites within which SNPs can be discovered. Modern array synthesis can generate on the order of 4 million specific sequences. This, in turn represents ˜0.1% of a human genome (3×10⁹ single strand, 6×10⁹ double strand), and ˜1% of the non-repeating portion of a human genome. Thus, this approach for generating the primer sets is the most direct way to identify SNPs in targeted regions of a human genome of approximately this size.

Superficially, this approach may resemble the Comparative Genome Sequencing (CGS) offered by NimbleGen. Here, arrays are synthesized to permit brute force re-sequencing (or survey re-sequencing) of entire microbial genomes. This is a brute force approach for identifying the locations of SNPs, insertions, or deletions. It is distinct from the instant invention by not involving the discovery of SNPs through the delivery of mixtures enriched in fragments that contain, or are adjacent to, SNPs. In the NimbleGen approach, both regions that contain SNPs and sequences that do not contain SNPs are re-sequenced.

Alternatively, split-and-pool methods can be used to generate libraries of oligonucleotides supported, for example, on beads. Then, the beads can be sorted based on their ability of the primers that they support to add a T, A, C, and G as the first nucleotide added when templated using the reference genome. Alternatively, the primers on the beads that, when templated using the reference genome, add three of the four standard nucleotides can be irreversibly blocked (using, for example, the 2′,3′-dideoxynucleoside triphosphates for the 3 nucleosides). This is limited by the number of beads that can be conveniently used (for example, a split and pool library that contains all 16-mers on average once requires approximately 4 billion beads). It does not require, however, knowledge of the sequence of the reference genome.

Alternatively, solution-based libraries constructed from random sequences can be prepared, and converted to the primer sets in four separate batches by templating these on multiple exemplars of the reference genome, where nucleotide N is added as the triphosphate at the same time as the triphosphates of the 3N nucleotides are added, where the products arising from the addition of N can be separated from the products arising from the addition of 3N, or where the products arising from the addition of 3N are irreversibly blocked from participating in the cleavage reaction that regenerates the N-extendable primer set, or irreversibly blocked from participating in another downstream process. As is understood by those skilled in the are, this has the advantage of not being limited by the number of beads that can be physically created, or the number of sequences that can be deliberately synthesized on (for example) a two dimensional array. It also does not require knowledge of the sequence of the reference genome.

1.2 Deriving the Primer Sets from the Physical DNA of the Reference Genome

Alternatively, the DNA from the reference genome can provide, physically, the DNA for the first step in the process. A simple approach to generate the four N-extendable primer sets involves treatment of the reference genome with restriction sites that leave a 3′-underhang where the complementary strand (now a 5′-overhang) templates the addition of N (T, A, C or G) as the first nucleotide in the extension reaction. This has the disadvantage of allowing the primer sets to query only those sites where a corresponding restriction enzyme can be found for use. This is, in turn, limited by the fact that most restriction sites that cleave within their recognition region have palindromic recognition sequences.

A fragmentation-polishing-ligation-digestion process that uses restriction endonucleases avoids this problem. Here, the DNA from the reference genome is physically fragmented and polished to give blunt end duplexes. These are then blunt-end ligated to duplexes that contain a restriction endonuclease site that, upon digestion, creates a 3′-underhang that adds just one of the four standard nucleotides.

An alternative approach generates libraries of 3′-underhangs from fragments of the reference genome. For example, in one such architecture, the reference genome is randomly fragmented to create duplexes. Partial digestion with 3′-exonuclease such as exonuclease III generates a library of underhang duplexes. These are processed by the addition of N and 3N to give differences that allow the separation of the products derived from N-addition and 3N-addition, or renders the products derived from 3N-addition inactive in downstream steps.

In all cases, primer extension times are short (ca. 2-15 seconds) to ensure that the only primers that are extended are those that have their 3′-ends perfectly matched. The capturable element is then used to capture the primers that have been extended. Then, the N₀—P₁ bond is cleaved in captured primers, creating the T-extendable primer set. Analogous processes generate the A-extendable, C-extendable, and G-extendable primer sets.

### 1.3 Procedures for Creating Primer Sets Using an Extension-Cutback Approach

In many architectures involving both synthetic DNA and processing of natural DNA, a sequence of steps involving the addition of N versus 3N followed by separation (which may not be necessary if the 3N extension products are rendered irreversibly inactive, for example by the addition of the 3N 2′,3′-dideoxynucleotides), requires the cutback of the added N to create a primer that can again add N when it is presented to the target genome.

Many procedures known in the art can be used for the cutback step.

1.3.1 Cutback when N is a Ribonucleoside

Both Joyce [Ast98][Joy97] and Patel and Loeb [Pat00] have described mutant Family A polymerases that add a ribonucleotide to the 3′-end of a primer. Ribonucleosides are added to the 3′-end of a DNA primer in a template-directed fashion by T7 RNA polymerase as well. When a set of primers (for example, derived in a solution library) is extended using the reference genome as the template (the reference genome being denatured by heating; it may also be fragmented), the ribonucleosides triphosphate for N, and the 2′-3′-dideoxyribonucleoside triphosphates for 3N, to generate the primer set for N, then all primers that added 3N are irreversibly terminated, while those that added N are terminated in a ribonucleoside or, if multiple additions ensued, by one or more N ribonucleosides eventually terminated in a dideoxyribonucleoside. If multiple additions ensued, treatment with ribonuclease A (RNase A) renders the primers that added N initially in the form where they have been extended by a single N-bearing ribonucleotide.

Treatment of this extended primer bearing a 3′-terminal N-ribonucleotide with sodium periodate at room temperature at neutral pH (the reaction is complete at 10 mM periodate in less than a minute) generates the 2′,-3′-diketone, which can be captured by imine formation, separating the primers that were extended through the addition of N from those that were extended through the addition of 3N, through the formation of an imine (for example) with a resin-bound amine, or as an oxime with a resin-bound O-alkoxylamine, or as a hydrazone using a resin-bound hydrazine. Then, using reactions known in the art [Bro53][Whi53], the ketone can be treated to suffer beta-elimination, releasing the original primer with a 3′-O-phosphate. Treatment of this mixture by alkaline phosphatase (resin bound, at pH 8) re-generates an extendable primer with the free 3′-OH. When done on the library, the product is a set of N extendable primers.

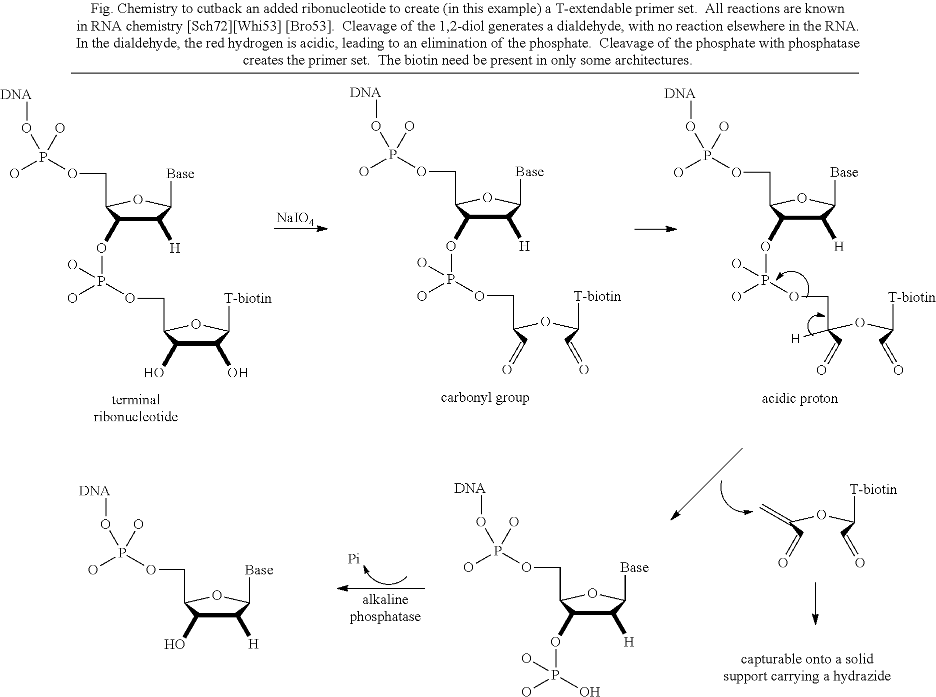

This cutback sequence can be used regardless of whether the primers are derived from chemical synthesis, or by fragmentation of the reference genome, or by 3′-exonuclease digestion, or by any other method.

1.3.2 Cutback when N is Preceded by a Ribonucleoside

When synthetic primers are used, or when messenger RNA is used as the source of reference material, the primers have a ribonucleotide already at their 3′-end. Addition of N as its alpha phosphorothioate nucleoside triphosphate, while addition of 3N as its 2′,3′-dideoxynucleoside triphosphate, permits a cutback process that works when N is added but not when 3N is added. This extension may be done by T7 RNA polymerase or, more preferably, by one of the DNA polymerases that accepts a 3′-ribonucleotide in its primer (e.g. Bst DNA polymerase, large fragment, Therminator, T7 DNA polymerase, T4 DNA polymerase, Klenow fragment, or phi29 DNA polymerase). This is based on the fact [Gis88] that treatment of a phosphorothioate that is preceded by a ribonucleosides with iodine (as an oxidizing agent) or with an alkylating agent (such as iodoethane) causes the cleavage via a 2′,3′-cyclic phosphate intermediate. The 3′-end of the primer is then restored by treatment with RNase A (which opens the 2′,3′-cyclic phosphate) followed by alkaline phosphatase.

### 1.4 Exploiting Capture Tags

In various of the architectures that implement the process of the instant invention, capture tags may be used. These may be used as a part of a required separation; separation is required when the 3N primers are not rendered permanently inactive in the generation of the N primer set. Alternatively, separation may be convenient to remove the 3N-extended primers even if they have been rendered permanently inactive, just to simplify downstream processing by hot having a substantial amount of unuseful DNA present.

In each of these cases, it is possible to replace the 2′,3′-dideoxynucleoside triphosphates by the commercially available 2′,3′-dideoxynucleoside triphosphates having a biotinylated capture tag, or the 2′,3′-dideoxynucleoside triphosphates having an alpha thiophosphodiester unit. This allows the primers that have been extended by a 3N triphosphate to be captured on an avidin or mercury column/beads (respectively).

Alternatively, the N nucleotide added may carry the capture tag.

### 2. Step 2. Using the Sets of Primer Sequences to Discover SNPs

The essence of the instant invention is to present, separately, the T-extendable primer sets, the A-extendable primer sets, the C-extendable primer sets, and the G-extendable primer sets, to the reference genome, and to apply procedures that deliver to a mixture of DNA fragments (single or double stranded) that is depleted in those that added T, A, C, and G respectively (that is, that added N) and enriched in those that added not-T, not-A, not-C, and not-G respectively (that is, that added 3N).

The extracted products have, therefore, discovered a SNP, a difference between the target and reference genomes. The discovery is realized by sequencing the primers, which allows us to identify the locus of the SNP in the genome.

Again, many architectures are taught by this disclosure that achieve this end. Fundamentally, they involve the addition of N and 3N that differ in a feature that permits them to be differentially separated, or differentially processed downstream. Several of these features (but not all) are outlined below, and presented in various examples. Additional features not presented can be imagined from the teachings in this disclosure by those skilled in the art.

In any library approach, the primers from Step 1 may find their own complements from the reference genome. This is not a big problem, in that these will in general not discover SNPs, and therefore will not enter the enriched sample.

In many architectures, the protocol of Step 2 include: (a) contacting in a solution where DNA duplexes can form each of the primer sets (each set individually) with the target genome, (b) providing a polymerase that extends those primers by adding a nucleotide to the primers under the direction of the hybridized templates from the target genome, and (c) separating the extended primer products that added the same nucleotide as would have been added had the reference genome provided the template from the extended primer products that added a different nucleotide. The separation step is not necessary if the products that have not discovered a SNP are rendered inactive in downstream processing.

This is the output of the instant invention, a collection of nucleotide fragments derived from the target genome enriched in those where the target genome sequence differs from the corresponding sequence in the reference genome. Then, a procedure is applied to obtain sufficient information from the second set of products (those that added a different nucleotide) to determine the location within the in silico genome of the sequence.

2.1 Exploiting Irreversibly Terminated Nucleoside Triphosphates for N in Competition with Reversibly Terminated Nucleoside Triphosphates for 3N

One direct way to distinguish between primers that added N from those that added 3N is to present N as its 2′,3′-dideoxynucleoside triphosphate, which generates an irreversibly terminated product, and 3N as the standard 2′-deoxynucleoside triphosphates, which are not immediately terminated. Depending on the length of the template, the primer extension will continue following the first addition until such time as the template calls for incorporation of an N nucleotide, or until the template ends. In the first case, the products (both those that found a SNP as well as those that did not) are irreversibly terminated, and cannot be easily processed.

One way to mitigate this is to present the 3N triphosphate with a 3′-reversible terminator. For example, the 3′-O-allyl-2′-deoxynucleoside triphosphates are incorporated by Therminator polymerase and its mutant forms and serve as reversible terminators, blocking extension until it is cleaved with a palladium catalyst [Seo05]. More preferably, the 3′-O—NH₂-2′-deoxynucleoside triphosphates [U.S. patent application Ser. No. 11/513,916] is incorporated with the Tabor-Richardson polymerase. It also blocks elongation, until it is removed by treating with acetate buffered sodium nitrite: HONO, preferably between pH 6 and pH 7, at room temperature, incubation preferably for less than 30 min.

With a reversible terminator, the terminator may be cleaved from the 3N-extended sequences that have discovered a SNP, under conditions where the N-extended sequences remain inert to further extension. Thus, after the terminating triphosphates are removed or destroyed, the 3N-extended sequences can be further extended on the template from the target genome, or ligated to another DNA sequence, which may be used to enter the 454 sequencing procedure, or used for PCR amplification.

For example, it is possible to deliver the output directly for 454 sequencing. In this case, the output is preferred to be double stranded, blunt ended, with all four ends chemically suited for ligation. Downstream 454 sequencing is particularly preferred when the output contains single exemplars of the sequences that have found SNPs.

### 2.2 Exploiting Differentially Terminating Nucleoside Triphosphates for N and for 3N

Different functionality on the 3′-position of the 3N-extended and the N-extended products may also be used to differentially deliver DNA fragments that have discovered a SNP. For example, one architecture presents N as its 2′,3′-dideoxynucleoside triphosphate and 3N as their 2′,3′-dideoxy-3′-aminonucleoside triphosphates. These are incorporated by polymerases known in the art [Tab95], with termination in both cases. Then, the 3′-amino group in the 3N-extended primers can be used to capture a downstream PCR primer binding site, a defined sequence that has a 5′-homologated (a DNA molecule that, at its 5′-end, has the 5′-OH group replaced by a CH₂CHO unit) or, preferentially, a 5′-bishomologated nucleoside (a DNA molecule that, at its 5′-end, has the 5′-OH group replaced by a —CH₂CH₂CHO unit) at its 5′-terminus. These form imines with the 3′-amino group of the 3N-extended primers that can be captured as the secondary amine through treatment with sodium cyanoborohydride at pH 6-8 in a process well known in the art. The downstream PCR primer binding site can be used to amplify the 3N-extended primers, to prepare them for sequencing. Many polymerases, including Taq and Therminator, read through this single unnatural secondary amine linkage in a template.

Alternatively, the homologated or bishomologated species may capture sequence that forms a hairpin. Especially in bead-bound libraries, these can be delivered directly to an Intelligent BioSystems instrument for sequencing.

### 2.3 Exploiting Differential Capture

Through the differential tagging of the N and 3N triphosphates, the 3N-extended and the N-extended products may be separated. For example, if the primer sets have a 3′-ribonucleosides, presenting the 3N-triphosphates as 2′,3′-dideoxynucleosides in a biotinylated form, but not having the N-2′,3′-dideoxynucleoside triphosphates biotinylated, the N-extended and the N-extended products may be separated on an avidin column. Then, for downstream sequencing, RNase cleavage will remove the 2′,3′-dideoxynucleoside tag, re-generating a ligatable 3′-terminus (necessary for the 454 sequencing pipeline).

### 2.4 Exploiting Differential Extendability

If the 3N-triphosphates may be presented as ribonucleosides triphosphates using the Joyce polymerase [Ast98], with the N-triphosphate presented as its 2′,3′-dideoxynucleoside, a single extension is achieved, with further extension possible by changing the polymerase to one that accepts a template having a ribonucleoside at its 3′-end.

### 3. Step 3. Determining the Location of the SNP

In all cases, the output of the second step is a collection of oligonucleotides enriched in those that have found a SNP, or enriched in DNA that can be downstream processed. The preferred form of that output depends on how, downstream, the information in that fragment will be used to place the SNP within the in silico genome.

In many architectures for downstream sequencing, including the 454 architecture, is possible that the downstream sequence will be determined by ligation of a sequencing primer to the 3′-end. If single molecules are delivered, then PCR amplification is desired. If, however, the fragments that have discovered a SNP are present on a bead made via split-and-pool, with enough copies to be directly sequenced (for example, on an Intelligent BioSystems instrument).

### REFERENCES

[All91] Allemann, R. K., Presnell, S. R., Benner, S. A. (1991) A hybrid of bovine pancreatic ribonuclease and angiogenin. An external loop as a module controlling substrate specificity? Prot. Engineering 4, 831-835
[Ast98] Astatke, M., Ng, K., Grindley, N. D., Joyce, C. M. (1998) A single side chain prevents Escherichia coli DNA polymerase I (Klenow fragment) from incorporating ribonucleotides. Proc. Natl. Acad. Sci. USA 95, 3402-3407.
[Ben00] Benner, S. A., Chamberlin, S. G., Liberles, D. A., Govindarajan, S., Knecht, L. (2000) Functional inferences from reconstructed evolutionary biology involving rectified databases. An evolutionarily-grounded approach to functional genomics. Research Microbiol. 151, 97-106
[Ben01a] Benner, S. A. (2001) Natural progression. Nature 409, 459
[Ben01b] Benner, S. A., Gaucher, E. A. (2001) Evolution, language and analogy in functional genomics. Trends in Genetics 17, 414-418
[Ben02] Benner, S. A., Caraco, M. D., Thomson, J. M., et al. (2002) Evolution—Planetary biology—Paleontological, geological, and molecular histories of life. Science 296, 864-868
[Ben03] Benner, S. A., Gaucher, E. A., Li, T. (2003) Post-genomic evolutionary analyses of the Severe Acute Respiratory Syndrome (SARS) virus genome using the MasterCatalog interpretive proteomics platform. Pharmagenomics—Application Notebook 2003, 23
[Ben04] Benner, S. A. (2004) Understanding nucleic acids using synthetic chemistry. Acc. Chem. Res. 37, 784-797
[Ben07] Benner, S. A., Sassi, S. O., Gaucher, E. A. (2007) Molecular paleosciences. Systems biology from the past. Adv. Enzymol. Related Areas Mol. Biol. Protein Evol. 75, 1-132 (Toone, E., ed.). Wiley, Chichester
[Ben07a] Benner, S. A. (2007) A method for sequencing DNA and RNA by synthesis. U.S. patent application Ser. No. 11/513,916
[Ben91] Benner, S. A., Gerloff, D. L. (1991) Patterns of divergence in homologous proteins as indicators of secondary and tertiary structure. The catalytic domain of protein kinases. Adv. Enzyme Regulat. 31, 121-181
[Ben92] Benner, S. A. (1992) Predicting de novo the folded structure of proteins. Curr. Opin. Struct. Biol. 2, 402-412
[Ben93b] Benner, S. A., Cohen, M. A., Gerloff, D. L. (1993) A predicted secondary structure for the Src homology domain 3. J. Mol. Biol. 229, 295-305
[Ben94] Benner, S. A., Badcoe, I., Cohen, M. A., Gerloff, D. L. (1994) Bona fide prediction of aspects of protein conformation. Assigning interior and surface residues from patterns of variation and conservation in homologous protein sequences. J. Mol. Biol. 235, 926-958
[Ben95] Benner, S. A., Gerloff, D. L., Chelvanayagam, G. (1995) The phospho-β-galactosidase and synaptotagmin predictions. Proteins. Struct. Funct. Genet. 23, 446-453
[Ben97] Benner, S. A., Turcotte, M., Cannarozzi, G., Gerloff, D. L., Chelvanayagan, G. (1997) Bona fide predictions of protein secondary structure using transparent analyses of multiple sequence alignments. Chem. Rev. 97, 2725-2843
[Bra05] Bradley, M. E., Benner, S. A. (2005) Phylogenomic approaches to common problems encountered in the analysis of low copy repeats: The sulfotransferase 1A gene family example. BMC Evolutionary Biology 5, Art. No. 22
[Bra06] Bradley, M. E., Benner, S. A. (2006) Integrating protein structures and precomputed genealogies in the Magnum database: examples with cellular retinoid binding proteins. BMC Bioinformatics 7, 89
[Bro53] Brown, D. M., Fried, M., Todd, A. R. (1953) The determination of nucleotide sequence in polyribonucleotides. Chem. Ind. (London) 352-353
[Cha04] Chang, M., Benner, S. A. (2004) Empirical analysis of insertions and deletions in protein sequence evolution. J. Mol. Biol. 341, 617-631
[Cho92] Chothia, C. (1992) One thousand families for the molecular biologist. Nature 357, 543-544
[Coh94] Cohen, M. A., Benner, S. A., Gonnet, G. H. (1994) Analysis of mutation during divergent evolution. The 400 by 400 dipeptide mutation matrix. Biochem. Biophys. Res. Comm. 199, 489-496
[DeF95] DeFay, T., Cohen, F. E. (1995) Evaluation of current techniques for ab initio protein structure prediction. Proteins 23, 431-445
[Dor90] Dorit, R. L., Schoenbach, L, Gilbert, W. (1990) How big is the universe of exons?Science 250, 1377-1382
[Elb04a] Elbeik, T., Surtihadi, J., Destree, M., Gorlin, J., Holodniy, M., Jortani, S. A., Kuramoto, K., Ng, V., Valdes, R., Valsamakis, A. et al. (2004) Multicenter evaluation of the performance characteristics of the Bayer Versant HCV RNA 3. 0 assay (bDNA) J. Clin. Microbiol., 42, 563-569
[Elb04b] Elbeik, T., Markowitz, N., Nassos, P., Kumar, U., Beringer, S., Haller, B. and Ng, V. (2004) Simultaneous runs of the Bayer Versant HIV-1 version 3.0 and HCV bDNA version 3.0 quantitative assays on the system 340 platform provide reliable quantitation and improved work flow. J. Clin. Microbiol., 42, 3120-3127
[Fah01] Faham, M., Baharloo, S., Tomitaka, S., DeYoung, J., Freimer, N. B. (2001) Mismatch repair detection (MRD): high throughput scanning for DNA variations. Human Mol. Genetics. 10, 1657-1664
[Fah05] Faham, M., Zheng, J. B., Moorhead, M., Fakhrai-Rad, H., Namsaraev, E., Wong, K., Wang, Z. Y., Chow, S. G., Lee, L., Suyenaga, K., Reichert, J., Boudreau, A., Eberle, J., Bruckner, C., Jain, M., Karlin-Neumann, G., Jones, H. B., Willis, T. D., Buxbaum, J. D., Davis, R. W. (2005) Multiplexed variation scanning for 1,000 amplicons in hundreds of patients using mismatch repair detection (MRD) on tag arrays. Proc. Natl. Acad. Sci. USA 102, 14717-14722
[Fak04] Fakhrai-Rad, H., Zheng, J. B., Willis, T. D., et al. (2004) SNP discovery in pooled samples with mismatch repair detection. Genome Res. 14, 1404-1412
[Fuk02] Fukami-Kobayashi, K., Schreiber, D. R., Benner, S. A. (2002) Detecting compensatory covariation signals in protein evolution using reconstructed ancestral sequences. J. Mol. Biol. 319, 729-743
[Gau01a] Gaucher, E. A., Das, U. K., Miyamoto, M. M., Benner, S. A. (2001) The crystal structure of eEF1a supports the functional predictions of an evolutionary analysis of rate changes among elongation factors. Mol. Biol. Evol. 19, 569-573
[Gau01b] Gaucher, E. A., Miyamoto, M. M., Benner, S. A. (2001) Function-structure analysis of proteins using covarion-based evolutionary approaches. Elongation factors. Proc. Natl. Acad. Sci. USA 98, 548-552
[Gau02] Gaucher, E. A., Gu, X., Miyamoto, M. M., Benner, S. A. (2002) Predicting functional divergence in protein evolution by site-specific rate shifts. Trends Biochem. Sci. 27, 315-321
[Gau03a] Gaucher, E. A., Thomson, J. M., Burgan, M. F., Benner, S. A. (2003) Inferring the paleoenvironment during the origins of bacteria based on resurrected ancestral proteins. Nature 425, 285-288
[Gau03b] Gaucher, E. A., Miyamoto, M. M., Benner, S. A. (2003) Evolutionary, structural and biochemical evidence for a new interaction site of the leptin obesity protein Genetics 163, 1549-1553
[Gau04] Gaucher, E. A., Graddy, L. G., Simmen, R. C. M., Simmen, F. A., Kowalski, A. A., Schreiber, D. R., Liberles, D. A., Janis, C. M., Chamberlin, S. G., Benner, S. A. (2004) The planetary biology of cytochrome P450 aromatase from swine. BMC Biology. 2, Art. No. 19
[Gau06] Gaucher, E. A., De Kee, D. W., Benner, S. A. (2006) Application of DETECTER, an evolutionary genomic tool to analyze genetic variation, to the cystic fibrosis gene family. BMC Genomics 7, Art. No. 44
[Gey03] Geyer, C. R., Battersby, T. R., Benner, S. A. (2003) Nucleobase pairing in expanded Watson-Crick like genetic information systems. The nucleobases. Structure 11, 1485-1498
[Gis88] Gish, G., Eckstein, F. (1988) DNA and RNA sequence determination based on phosphorothioate chemistry. Science 240, 1520-1522.
[Gon00] Gonnet, G. H., Korostensky, C., Benner, S. A. (2000) Evaluation measures of multiple sequence alignments. J. Comput. Biol. 7, 261-276
[Gon91] Gonnet, G. H., Benner, S. A. (1991) Computational Biochemistry Research at ETH. Technical Report 154, Departement Informatik, March
[Gon92] Gonnet, G. H., Cohen, M. A., Benner, S. A. (1992) Exhaustive matching of the entire protein sequence database. Science 256, 1443-1445
[Gon93] Gonnet, G. H., Benner, S. A. (1993) A word in your protein. Nature 361, 121
[Hod07] Hodges E, Xuan Z, Balija V, et al. (2007) Genome-wide in situ exon capture for selective resequencing. Nature Genetics 39, 1522-1527
[Joh04] Johnson, S. C., Marshall, D. J., Harms, G., Miller, C. M., Sherrill, C. B., Beaty, E. L., Lederer, S. A., Roesch, E. B., Madsen, G., Hoffman, G. L., Laessig, R. H., Kopish, G. J., Baker, M. W., Benner, S. A., Farrell, P. M., Prudent, J. R. (2004) Multiplexed genetic analysis using an expanded genetic alphabet. Clin. Chem. 50, 2019-2027
[Joy97] Joyce, C. M. (1997) A single side chain prevents Escherichia coli DNA polymerase I (Klenow fragment) from incorporating ribonucleotides. Proc. Natl. Acad. Sci. USA 94, 1619-1622
[Kim08] Kim, H-J., Kim, M-J., Karalkar, N., Hutter, D., Benner, S. A. (2008) Synthesis of pyrophosphates for in vitro selection of catalytic RNA molecules. Nucleosides, Nucleotides and Nucleic Acids 27, 43-56
[Lea06] Leal, N. A., Sukeda, M., Benner, S. A. (2006) Dynamic assembly of primers on nucleic acid templates. Nucleic Acids Res. 34, 4702-4710
[Lee07] Lee, W-M., Grindle, K., Pappas, T., Marshall, D. J., Moser, M. J., Beaty, E. L., Shult, P. A., Prudent, J. R., Gem, J. E. (2007) High-throughput, sensitive, and accurate multiplex PCR-microsphere flow cytometry system for large-scale comprehensive detection of respiratory viruses. J. Clin. Microbiol. 45, 2626-2634
[Li06] Li, T., Chamberlin, S. G., Caraco, M. D., Liberles, D. A., Gaucher, E. A., Benner, S. A. (2006) Analysis of transitions at two-fold redundant sites in Mammalian genomes. Transition redundant approach-to-equilibrium (TREx) distance metrics. BMC Evolutionary Biol. 6, 25
[Lib01] Liberles, D. A., Schreiber, D. R., Govindarajan, S., Chamberlin, S. G., Benner, S. A. (2001) The adaptive evolution database (TAED) Genome Biol. 2, 0003.1-0003. 18
[Mes97] Messier, W., Stewart, C. B. (1997) Episodic adaptive evolution of primate lysozymes. Nature 385, 151-154
[Pat00] Patel, P. H, Loeb, L. A. (2000) Multiple amino acid substitutions allow DNA polymerases to synthesize RNA. Proc. Natl. Acad. Sci. 275, 40266-40272
[Pel00] Peltier, M. R., Raley, L. C., Liberles, D. A., Benner, S. A. Hansen, P. J. (2000) A comprehensive evolutionary analysis and structural prediction of the uterine serpin family. J. Exp. Zool. (Mol. Devel. Evol.) 288, 165-174
[Pet07] Peters, B. A., Kan, Z. Y., Sebisanovic, D., et al. (2007) Highly efficient somatic mutation identification using Escherichia coli mismatch-repair detection. Nature Methods 4, 713-715
[Sac01] Sachidanandam, R., Weissman, D., Schmidt, S. C., Kakol, J. M., Stein, L. D., Marth, G., Sherry, S., Mullikin, J. C., Mortimore, B. J., Willey, D. L., et al. 2001. A map of human genome sequence variation containing 1.42 million single nucleotide polymorphisms. Nature 409, 928-933
[Sas07a] Sassi, S. O., Braun, E. L., Benner, S. A. (2007) The evolution of seminal ribonuclease: Pseudogene reactivation or multiple gene inactivation events? Mol. Biol. Evol. 24, 1012-1024
[Sas07b] Sassi, S. O., Benner, S. A. (2007) The resurrection of ribonucleases from mammals. From ecology to medicine. Experimental Paleogenetics, D. A. Liberles, ed., NY, Academic Press
[Seo05] Seo, T. S., Bai, X., Kim, D. H., Meng, Q., Shi, S., Ruparel, H., Li, Z., Turro, N. J., and Ju, J., (2005) Four-color DNA sequencing by synthesis on a chip using photocleavable fluorescent nucleotides. Proc. Natl. Acad. Sci. 102, 5926-5931
[Sjo08] Sjoblom, T. (2008) Systematic analyses of the cancer genome: lessons learned from sequencing most of the annotated human protein-coding genes. Curr. Opin. Oncol. 20, 66-71
[Tab95] Tabor, S., Richardson, C. C. (1995) A single residue in DNA-polymerases of the Escherichia coli DNA-polymerase I family is critical for distinguishing between deoxyribonucleotides and dideoxyribonucleotides. Proc. Natl. Acad. Sci. USA 92, 6339-6343
[Tau97] Tauer, A., Benner, S. A. (1997) The B12-dependent ribonucleotide reductase from the archaebacterium Thermoplasma acidophila. An evolutionary conundrum. Proc. Nat. Acad. Sci. 94, 53-58
[Tho05] Thomson, J. M., Gaucher, E. A., Burgan, M. F., De Kee, D. W., Li, T., Aris, J. P., Benner, S. A. (2005) Resurrecting extinct proteins from ancient yeast at the origin of fermentation. Nature Genetics 37, 630-635
[Top07] Topol, E. J., Frazer, K. A. (2007) The resequencing imperative. Nature Genetics 39, 439-440
[Tra96] Trabesinger-Ruef, N., Jermann, T. M., Zankel, T. R., Durrant, B., Frank, G., Benner, S. A. (1996) Pseudogenes in ribonuclease evolution. A source of new biomacromolecular function? FEBS Lett. 382, 319-322
[Wan80] Wangyi, Li., Xiangrong, G., Ju'e, C. (1980) A new fluorescent hydrazide for sequencing ribonucleic acids. Scientia Sinica 23, 1296-1308
[Wei91] Weinhold, Glasfeld, Ellington, Benner (1991) Structural determinants of stereospecificity of alcohol dehydrogenase. Proc. Nat. Acad. Sci. 88, 8420-24
[Whi53] Whitfield, P. R., Markham, R. (1953) Natural configuration of the purine nucleotides in ribonucleic acids. Chemical hydrolysis of the dinucleoside phosphates. Nature 171, 1151-1152
[Yan06] Yang, Z., Hutter, D., Sheng, P, Sismour, A. M., Benner, S. A. (2006) Artificially expanded genetic information system: A new base pair with an alternative hydrogen bonding pattern. Nucl. Acids Res. 34, 6095-6101
[Yan07a] Yang, Z., Sismour, A. M., Sheng, P., Puskar, N. L., Benner, S. A. (2007) Enzymatic incorporation of a third nucleobase pair. Nucl. Acids Res. 35, 4238-4249
[Yan07b] Yang, Z. Sismour, A. M., Benner, S. A. (2007) Nucleoside alpha-thiotriphosphates, polymerases and the exonuclease III analysis of oligonucleotides containing phosphorothioate linkages. Nucl. Acids Res. doi: 10.1093/nar/gkm168

### EXAMPLES

### Example 1

### The Ligate-Cleave Procedure to Generate Primer Sets

In addition to creating primer sets by synthesis of specific DNA sequences or by sorting DNA of random sequence, primers can be generated from the reference genome DNA itself (the physical DNA). This includes shearing the physical DNA (by sonication or focused sonication), by restriction endonucleases, by culling back with an exonuclease, ITCHY technologies, or other ways of creating truncated fragment libraries that are well known in the art.

This example illustrates the use of blunt end ligation followed by restriction endonuclease fragmentation to give, with three known restriction endonuclease, three of the fragments. First, the DNA from the reference genome is fragmented to fragments that are preferably 50-1000 nucleotides in length. The Covaris instrument in the art is known to provide such lengths, with shorter lengths arising from longer Covaris treatment. The ends of the fragments are made blunt ended (“polished”) by treatment with an exonuclease and, more preferably, by filling in with a DNA polymerase and 2′-deoxynucleoside triphosphates. The result is a collection of DNA duplex sequence fragments, with both ends being blunt and having, at each end, one of these four types of blunt end:

| | | | |
|---|---|---|---|
| DNA-T-3′ | DNA-C-3′ | DNA-G-3′ | DNA-A-3′ |
| DNA-A-5′ | DNA-G-5′ | DNA-C-5′ | DNA-T-5′ |

Where the DNA sequences are complementary. The following duplex sequence, prepared by Integrated DNA Technologies (IDT, Coralville) is then attached by blunt-end ligation to each end:

ATCNNNNN-3′-biotin
TAGNNNNN-5′

where N is any nucleotide (but the N's paired in the duplex are, of course, Watson-Crick complementary), and the oligo N segment is long is needed to get the ligation to go efficiently, preferably at least 10 nucleotides in length, with a sequence chosen to avoid Mbo1 sites and/or to facilitate downstream cloning. This gives the following products.

| | | | |
|---|---|---|---|
| (a) | DNA-TATCNNNNN-3′- | (b) | DNA-CATCNNNNN-3′- |
| | biotin | | biotin |
| | DNA-ATAGNNNNN-5′ | | DNA-GTAGNNNNN-5′ |
| (c) | DNA-GATCNNNNN-3′- | (d) | DNA-AATCNNNNN-3′- |
| | biotin | | biotin |
| | DNA-CTAGNNNNN-5′ | | DNA-TTAGNNNNN-5′ |

This creates a restriction site in only case (b); the sequence GATC is the recognition sequence for the restriction enzyme Mbo1. All of the other fragments do not contain sites for this enzyme. Digestion of all possible structures with Mbo1 removes the biotin only from the top strand of (c) to give as the only fragments that do not retain their ability to bind to streptavidin, in the following form:

DNA-
DNA-CTAG

Because of the specificity of the Mbo1 restriction endonuclease, the DNA fragments that do not bind to streptavidin will all, when templated on the reference genome, will all add G first. Analogous separation can be done using other tags (for example, a thiol group at the 3′-will allow a mercury gel or column to separate the non-cleaved fragments from the cleaved fragments). Thus, the DNA fragments recovered from a streptavidin separation step is a set of G-extendable primers from this reference genome. It should be noted that to have utility, this process need not identify every G-extendable primer within the reference genome. Even as little as 20% coverage of the non-repeating regions has utility, more preferably 50% coverage of the non-repeat regions.
The same process is repeated to generate the A-extendable primer set, but with Tsp509I as the restriction endonuclease, with blunt end ligation to the following sequence where the length of the N region and its sequence is chosen as before:

| ATTNNNNN-3′-biotin | | | |
|---|---|---|---|
| TAANNNNN-5′ | | | |
| (a) | DNA-TATTNNNNN-3′- | (b) | DNA-CATTNNNNN-3′- |
| | biotin | | biotin |
| | DNA-ATAANNNNN-5′ | | DNA-GTAANNNNN-5′ |
| (c) | DNA-GATTNNNNN-3′- | (d) | DNA-AATTNNNNN-3′- |
| | biotin | | biotin |
| | DNA-CTAANNNNN-5′ | | DNA-TTAANNNNN-5′ |

This creates a restriction site in only one case, the one that is underlined, for the four cutter Tsp509I. All of the other fragments do not contain sites for this enzyme. Digestion with Tsp509I releases the biotin from (d) to generate:

DNA-
DNA-TTAA

Now, the only fragments that do not retain their ability to bind to streptavidin (after the duplex is denatured) will be extended by A when templated on the reference genome. This is therefore a set of A-extendable primers.
The C-extendable primer set is then prepared from the reference genome using StyD41, with blunt end ligation to the following sequence (N's defined as before):

CNGGNNNNN-3′-biotin
GNCCNNNNN-5′

Again where N is any nucleotide. The products are as follows:

| | | | |
|---|---|---|---|
| (a) | DNA-TCNGGNNNNN-3′- | (b) | DNA-CCNGGNNNNN-3′- |
| | biotin | | biotin |
| | DNA-AGNCCNNNNN-5′ | | DNA-GGNCCNNNNN-5′ |
| (c) | DNA-GCNGGNNNNN-3′- | (d) | DNA-ACNGGNNNNN-3′- |
| | biotin | | biotin |
| | DNA-CGNCCNNNNN-5′ | | DNA-TGNCCNNNNN-5′ |

This creates a restriction site in only one case, the one that is underlined, for the four cutter StyD41. All of the other fragments do not contain sites for this enzyme. Digestion with StyD41 gives the following unlabeled fragments that do not bind streptavidin, and which collectively make a C-extendable primers C:

DNA-
DNA′GGNCC

No restriction endonuclease is commercially available to create a T-extendable primer set. Nevertheless, these can be obtained upon recognizing that a T-extendable primer will be derived from one of four classes of blunt ends:

| | | | | | | | |
|---|---|---|---|---|---|---|---|
| (a) | DNA- | (b) | DNA- | (c) | DNA- | (d) | DNA- |
| | AT-3′ | | TT-3′ | | GT-3′ | | CT-3′ |
| | DNA- | | DNA- | | DNA- | | DNA- |
| | TA-5′ | | AA-5′ | | CA-5′ | | GA-5′ |

To generate the T-extendable primers, the fragment ligated must generate a restriction endonuclease site that cuts between the last and next to last sites. Thus, for (b), blunt end ligation of (b) to the segment (left) (N's defined as above) creates the product (middle) with the TTAA recognition site for the MseI enzyme (T*TAA, where * indicates the site of cleavage) generating a T-extendable fragment (right) following MseI cleavage.

| | | |
|---|---|---|
| AANNNNN-3′- | DNA-TTAANNNNN-3′- | DNA-T-3′ |
| biotin | biotin | |
| AANNNNN-5′ | DNA-AATTNNNNN-5′ | DNA-AAT-5′ |

For case (c), the ApaL1 enzyme is used (with the recognition sequence G*TGCAC) with the duplex for blunt end ligation, the product of that ligation, and the product following cleavage with ApaL shown below:

| | | |
|---|---|---|
| GCACNNN-3′-biotin | DNA-GTGCACNNN-3′- | DNA-G-3′ |
| | biotin | |
| CGTGNNN-5′ | DNA-CACGTGNNN-5′ | DNA-CACGT-5′ |

For case (d), the AflII enzyme is used (with the recognition sequence C*TTAAG) with the duplex for blunt end ligation, the product of that ligation, and the product following cleavage with AflII shown below:

| | | |
|---|---|---|
| TAAGNNN-3′- | DNA-CTTAAGNNN-3′- | DNA-C-3′ |
| biotin | biotin | |
| ATTCNNN-5′ | DNA-GAATTCNNN-5′ | DNA-GAATT-5′ |

As is appreciated by one of ordinary skill in the art, other restriction endonucleases exist that can be substituted for the enzymes listed above to generate the same outcome. These may be preferable depending on the methylation of the reference genome. Further, to prevent concatenation in the blunt end ligation, it is preferred that the synthetic short ligation fragments be blocked at their 3′-ends by a dideoxynucleotide. It should be noted that if this is done, the four N-extendable primer sets can be prepared without an absolute need for a separation, as the fragments that are not cleaved to not have a polymerase-active 3′-end. Thus, they cannot interfere with the second step in the process of the instant invention. No restriction endonuclease is commercially available to create the T-extendable primer sets.

### Example 2

### Immobilized Primer Sets

An alternative approach to generating the primer sets begins with the preparation of all possible primer sequences, followed by the use of the physical DNA from a reference genome to direct them into each of the four primer sets. The first example of this uses split-pool synthesis on beads, and a process that separates the bead-supported primers into the four sets set. One attribute of this particular architecture is that it allows, up front, an additional subtractive process that removes primers that prime on repeats. One invention for doing so is embedded into this example.

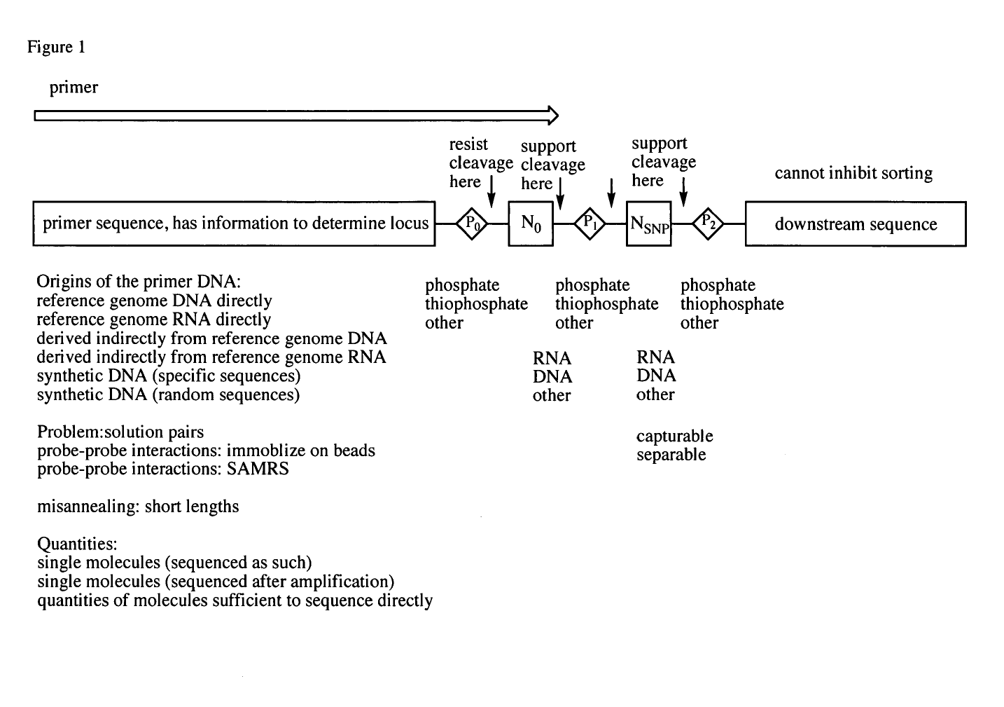

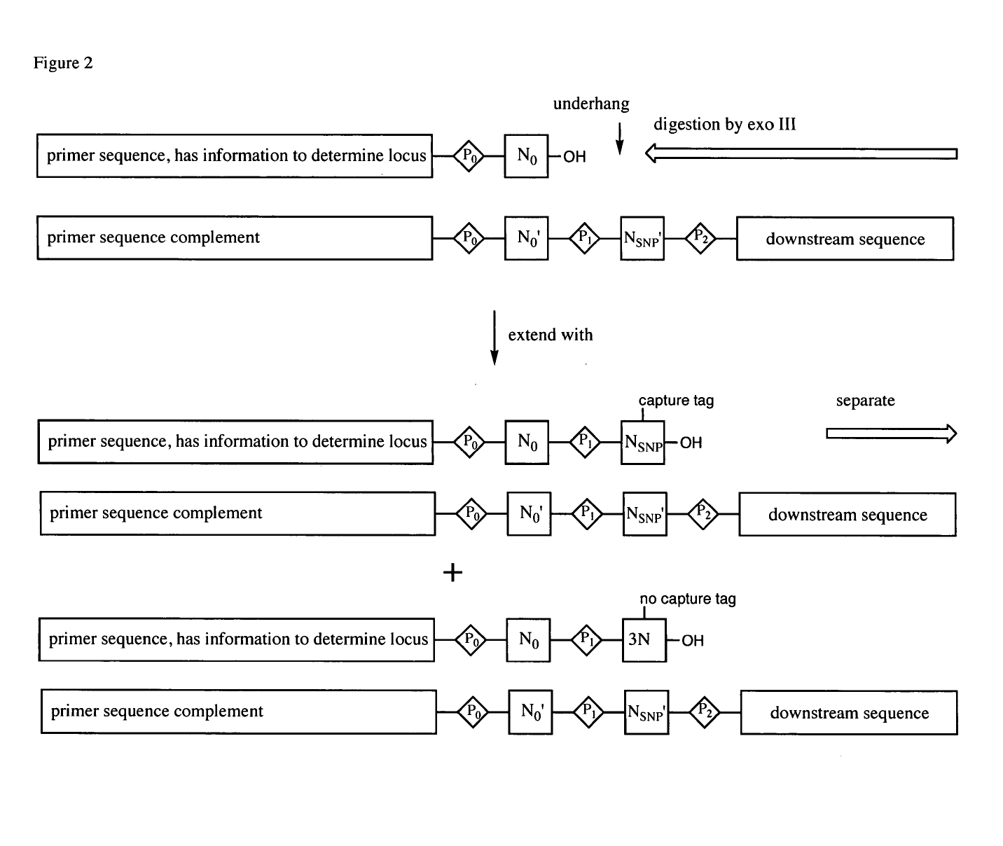

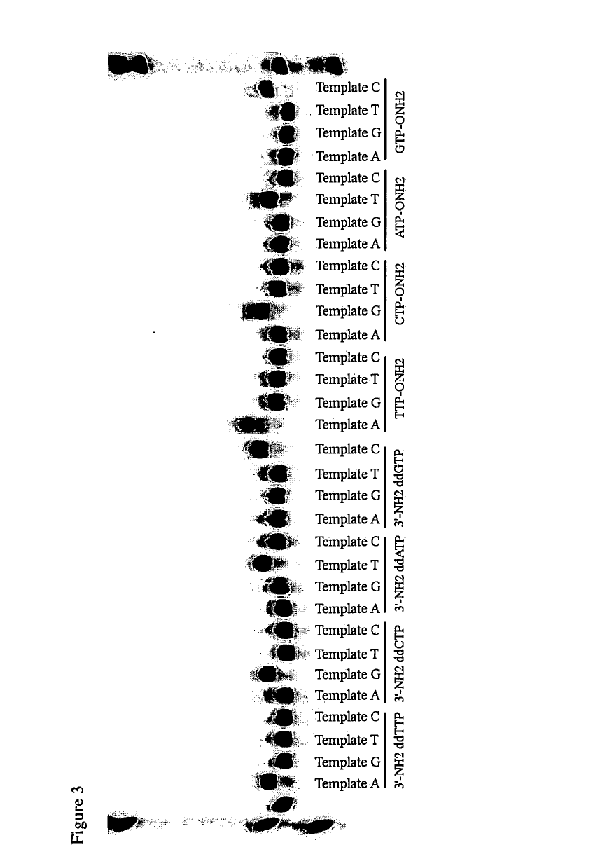

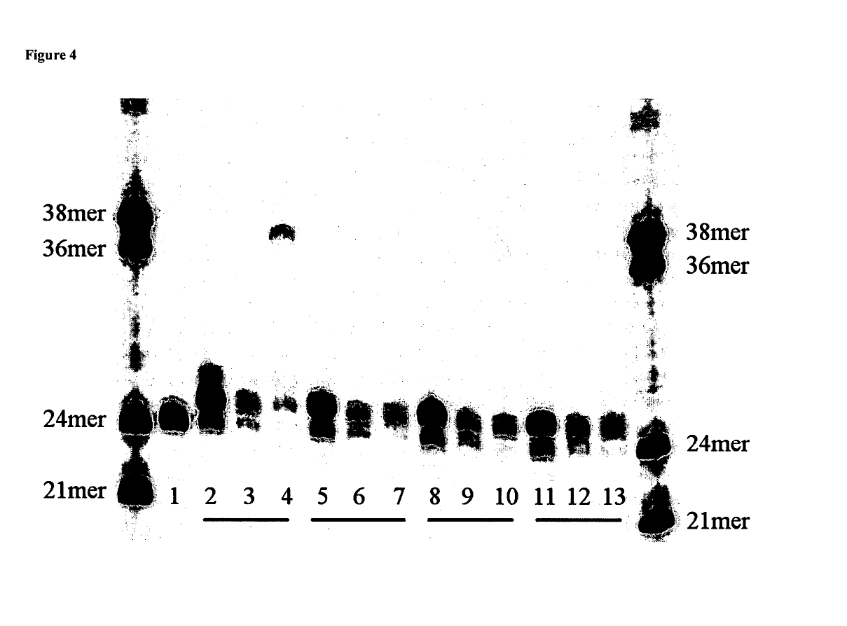

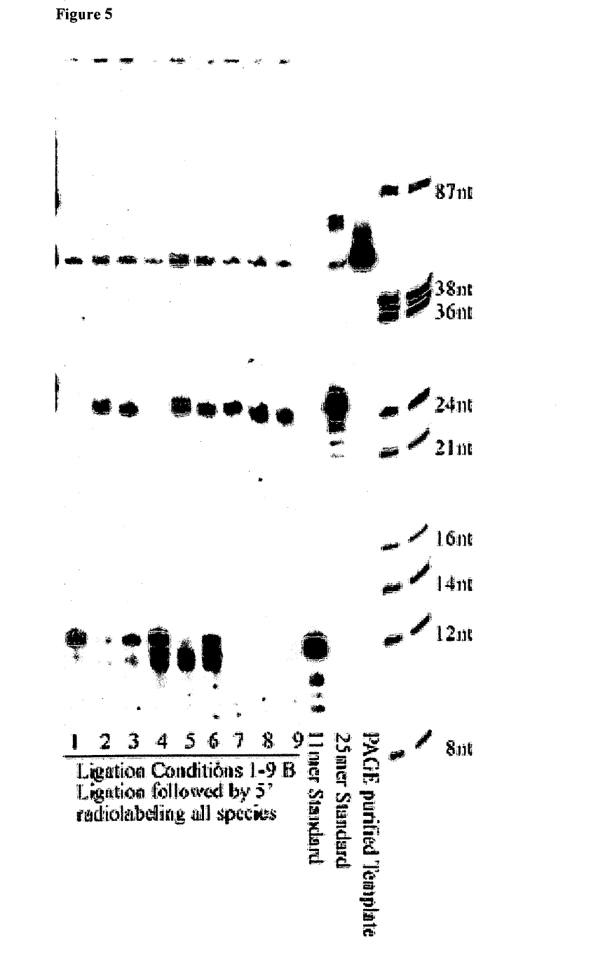

The work flowchart for a subtractive sequencing architecture is summarized in FIG. 4.1.

### Procedure 1. Prepare the Beads Carrying a Library

The most general architecture has a primer for every site. Considering the human genome as representative of a large genome, discovering SNPs throughout formally requires ca. 6×10⁹ primers (counting both strands). This is approximately all 16 mers built from four different nucleotides. This calculation assumes, of course, that the human genome has a random sequence. It does not, of course, and this fact is managed below.

Synthesis on the beads is done using the split and pool methodology. The beads may be non-porous, 4.5±0.3 microns in diameter made from highly crosslinked polymethylmethacrylate (PMMA), although any bead used in the art for split-and-pool synthesis of DNA may be used. Short PEG (polyethylene glycol) linkers (average molecular weight 1000, approximately 25-CH₂CH₂—O units) are attached before the synthesis (approximately 10⁵ PEGs per bead), with the synthesis then occurring at the end of the PEG. The PEG linker enables efficient DNA synthesis. Further, the hydrophilic nature of the PEG linker permits the attached synthetic DNA to later protrude into aqueous solution, where it has access to DNA from the reference and target genomes, as well as access to polymerases and restriction endonucleases.

Synthesis in this architecture is done in the atypical 5′-to-3′-direction using 3′-DMT protected 5′-phosphoramidites, which are commercially available (Berry, Glen). Diaminopurine and 5-propynyluracil may be used to replace adenine and thymine to make melting temperatures between the bead-supported primers and the genomic templates in solution more uniform. Approximately 10¹⁹ beads (about 1 mL in volume) are used. The 16 mer random region is preceded by a constant region that holds a primer binding site preceded and followed by a unique restriction endonuclease site; this is done without splitting and pooling the beads. The split and pool technology ensures that every random sequence on a single bead is identical to every other on the same bead. Between the beads, however, each 16 mer is found on average on 1.5 beads. Each 14 mer (which is expected to be sufficient to identify a locus in the non-repeating region in ca. 80% of the events) is represented at the 3′-end of the bead-bound synthetic sequence approximately 40 times. If desired, the diversity of the library may be established by releasing sequences from a bead and submitting a sample of these for 454 sequencing.

Choosing a 16 nt stretch as the random region is conveniently consistent with both biophysics and current technology. On average, hybridization of a 16 mer in the non-random region of the human genome must be selective against 1.8 mismatches, and therefore will discriminate against the second best hybridization partner in a genome about 80% of the time. The use of diaminopurine and propynylU as replacements for adenine and thymine helps this, as is well known in the art.

Procedure 2. Capping Primers that Target within Repeats

Depending on what one views as a repeat, approximately 90% of the human genome is repetitive, and 90% of the “high information” remainder is presently viewed as non-coding. The remaining “high information” portion, ca. 1% of the total human genome, is viewed as the “exome” that RFA-HL-08-004 proposes to target.

Repeats can raise the cost of a differential sequencing strategy by generating a large number of pseudo-polymorphisms that arise from inter-locus comparisons. Further, even if SNPs in the repeating region are discovered and cataloged, a 16 nt tag will generally be insufficient to allow their locus in the in silico genome to be determined.

An invention allows, however, the beads that carry primers that target repeat regions to be removed before the primer sets themselves are constructed. Here, the beads with synthetic templates are incubated with a set of synthetic DNA molecules having the repeat sequences obtained from the in silico genome. Following a contacting step (the beads are contacted with a solution containing the synthetic repeats), the mixture is incubated with fluorescently tagged 2′,3′-dideoxynucleoside triphosphates and the Tabor-Richardson variant of Taq DNA polymerase (which accepts 2′,3′-dideoxynucleoside triphosphates efficiently) [Tab95]. Any bead containing a primer that primes on the repeats will become fluorescent, and the primers that it carries will be irreversibly blocked. These bead are then separated from the beads carrying primers that do not prime on repeating elements using a Cytopeia cell sorter. The Cytopeia bead sorter sorts 70,000 beads per second, allowing bead sorting to be complete in 24 hours (70,000×60×60×24≈6×10⁹).

It is important to understand how incomplete reaction will influence the outcome of this procedure. With 10⁵ oligonucleotides on each bead, and a bead sorter able to detect beads with fewer than 1000 fluors, the sorting process can discriminate beads that are extended to any level of completion. Conversely, through mismatched priming, a small number of the beads that are not formally complementary to the repeat region will also be capped. Thus, there is a tradeoff. Addition of more repeating units to remove more beads will reduce cost-per-utility (by having a higher enrichment of the non-repeating SNPs) while decreasing coverage. Initially, the loss of coverage is of no concern, as the undiscovered SNPs are in the repeating regions, largely uninteresting from a biomedical perspective and impossible to locate within the in silico genome in any case.

Increasing the aggressiveness with which repeats are removed will, of course, eventually diminish the potential of the system to detect polymorphisms in the non-repeating regions. First, the ability of the subtractive sequencing tool to identify SNPs that follow a 16 gram found in a repeat region will be halved. This need not defeat the subtractive sequencing approach; such SNPs will, of course, be picked up in the antisense strand, unless that SNP has the double misfortune of following a 16 mer found in the repeat region in both strands. Analysis of the in silico genome will identify those pathological cases, as well as to explain why a SNP is found in only one strand. Thus, optimization of this step in the pipeline will combine experimental and bioinformatic work.

### Procedure 3. Extending the Templates Using 77 RNA Polymerase

The beads that are not capped by templating on repeats are then incubated with reference genome DNA as template. The reference genome is first fragmented using a Covaris instrument to generate fragments ca. 200 nts in length. Other fragmentation procedures known in the art may be used. Then, the 3′-ends of the fragments are capped by incubating them with by an unlabelled 2′,3′-dideoxynucleoside triphosphate and terminal transferase. This renders all of the template molecules inactive as primers, saving reagents, lowering background, and simplifying analysis.

The ends of the primer are then annealed to the fragments of the target genome, with the stringency of the annealing conditions adjusted by procedures well known in the art. Once annealed, the primers are extended by T7 RNA polymerase, using four ribonucleoside triphosphates, each bearing a fluor with a different emission color (FIG. 4.2). Experiments confirmed the ability of T7 RNA polymerase to execute this extension with a DNA primer and the tagged triphosphates. Extension appends an RNA tail to the primers that have found their template. Each nucleotide carries a fluor, however, meaning that the efficiency of extension will decrease as the tails become longer. The ratio of triphosphate is adjusted to make tail lengths short, preferably ca. 5 nucleotides.

### Procedure 4. Digesting Back the RNA Tails

The bead-supported extended products are then digested with ribonuclease (RNase) A (FIG. 4.2). This enzyme requires that the nucleotide ahead of the cleaving phosphodiester bond be a ribonucleotide, as the 2′-OH group participates in the mechanism of the reaction to form a cyclic phosphate as an intermediate. This means that only the first ribonucleotide will remain on the extended primer, and the bead will fluoresce with a color characteristic of that side chain. This means that the fluorescent color of the bead will be determined by the ribonucleotide that was first appended to the primer.

Procedure 5. Sorting the Beads to Separate Those that Added A, T, G, and C from Each Other.

The beads are then sorted by color using the Cytopeia cell (bead) sorter, using the four color sorting feature of the instrument, reflecting the fact that the beads whose primers have been extended with A, T, G, or C have different colors. The sorter separates ca. 70,000 beads per second. Since the repeats (and 90% of the beads) have been removed, the sorting time is ca. 2.4 hours.

A fraction of the primers on the beads will, of course, be extended with templates that annealed to form duplexes with 1-2 mismatches. This will not defeat the separation process, as the efficiency of extension will drop (depending on the annealing and extension recipes) by at least a factor of 10 for a single mismatch, and another factor of 10 for a double mismatch. Thus, the blue color (>90%) might be diluted with (for example) red (<9%) and cyan (<<1%), but this can be managed by adjusting the thresholds on the bead sorter. More desirably, however, the annealing and extension recipes will be adjusted.

Procedure 6. Recover Beads that Incorporate More than One Nucleotide in ˜50:50 Ratio

Two features of the reference genome have the potential for complicating sorting. If the reference genome is heterozygous, then a fraction of the beads will have two colors attached in approximately a 1:1 ratio. The extent of heterozygosity depends, of course on the number of SNPs that separate the father and the mother of the individual providing the reference genome. This is the same number of SNPs that separate two individuals in the same breeding population generally.

The ability of the cell sorter to deliver beads that capture two colors provides us with an opportunity to analyze the genotype of the well-phenotyped individual. The beads that have captured two different colors in a 1:1 ratio are binned, and passed directly to the sequencing phase of the process. This identifies the loci at which those individuals are heterozygous, itself useful information.

Low copy number repeats also create apparent heterozygosity. Such repeats in sulfotransferases were recently analyzed by the Benner group from a bioinformatics perspective in humans and chimpanzees [Bra05]. As sulfotransferases are used to detoxify drugs, “polymorphism” between these loci may also correlate with the outcome of particular therapeutic regimens. As the in silico genome also records what genes are repeated in low copy numbers, these will be directly identified by sequencing the random regions.

### Procedure 7. Create Beads Holding T-Extendable, A-Extendable, C-Extendable, and G-Extendable Primers

To complete the first protocol, the final ribonucleotide is cut from each member of each of the extended primers. This is done using the sequence whose chemistry is shown above. Periodate treatment (5 min, 25° C.) at pH 8.4 in tetramethylglycinamide buffer cleaves the 2′-3′-bond of the ribonucleoside to give the dialdehyde [Sch72][Whi53][Bro53]. This renders acidic the 4′-proton (adjacent to a carbonyl C═O unit): It is well known from classical RNA chemistry that upon incubation at 45° C. for 2 hours, cleavage is complete. Using a hydrazide as a cleaving reagent [Wan80] allows the small fragment to be captured. The 3′-end a phosphate group is removed using alkaline phosphatase, restoring the 3′-OH to generate the T-specific primer set. The A-extendable, C-extendable, and G-extendable primers sets are then obtained by analogous reactions.

### Distinguishing SNP and Non-SNP Extension Product Using the Bead-Bound Primers.

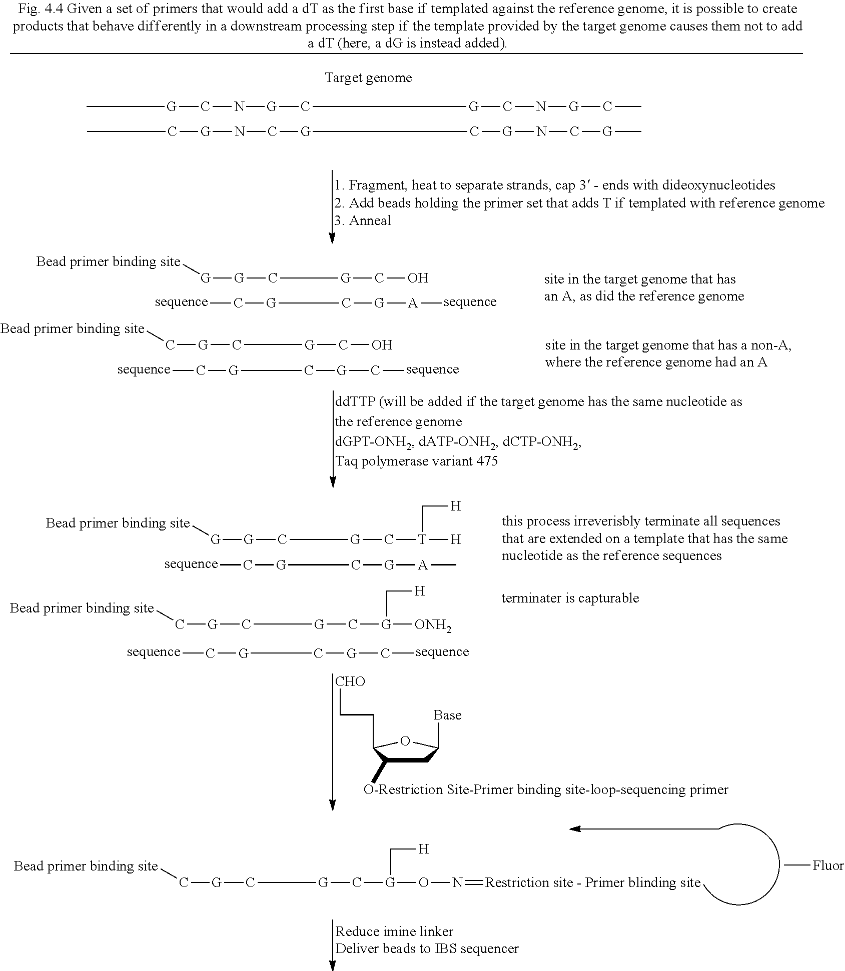

On any set of beads arising from the first protocol, the primers will all add the same nucleotide when templated with the reference genome. Thus, to identify sites in the target genome that differ from the analogous sites in the reference genome, a second step must be used to identify beads whose primers would be extended by nucleotide p if targeted against the reference genome as template, but are extended by nucleotide q when targeted against the target genome as template. Again, many architectures can do this; the following architecture uses the libraries prepared on beads described.

Each set of beads are separately contacted to a sample of the target genome, which has been Covaris fragmented (preferably the fragments are between 50 and 200 nucleotides) and capped as described above for the reference genome. The fragments of the target genome provide the templates for the second extension, and a dideoxynucleoside triphosphate (for the nucleotide called for by the non-SNP template) is used to cap the primers if they are extended by the same nucleotide as would be called for by the reference genome template. Thus, for the beads holding the T-extendable primers, dideoxyTTP is used to cap the primers that do not detect a SNP. For beads holding A-extendable primers, ddATP is used to cap the primers that do not detect a SNP. For beads holding G-extendable and C-extendable primers, ddGTP and ddCTP (respectively) are used to cap the primers that do not detect a SNP.

At the same time, the other three deoxynucleoside triphosphates are introduced in a form where the nucleoside carries a 3′-ONH₂ unit that permits separation and/or primer extension, and/or cloning.

### Use of the 3′-ONH₂ Unit.

This unit is a reversible terminator [Ben07a] that is incorporated by DNA polymerases, including the Tabor-Richardson version of Taq polymerase, and a variant of the Taq polymerase where the following sites are changed: E517G, K537I, L613A. The product mixture is then treated with HONO. In. Once incorporated, further extension is blocked. Once incorporated, the 3′-ONH₂ unit may be used as a handle to capture a downstream oligonucleotide (FIG. 4.4). Alternatively, it can be removed by treatment with dilute HONO-nitrate buffer (preferably at pH 5-7) to regenerate a free, extendable, 3′-OH group. This also converts any of the triphosphates. Addition of excess of the standard triphosphate to overwhelm the nucleoside triphosphate Thus, polymerase extension on the same template can continue, but only in the case here a 3N nucleotide (meaning one of the three nucleotides that is not the nucleotide that would be added in if reference genome provided the template).

The principal difficulty is that the irreversible terminator remains in the pool. This can either be destroyed by a phosphatase, with the phosphatase then being destroyed before a fresh set of elongating triphosphates is added, or it can be overwhelmed by adding large excess of the new triphosphates, or the duplex can be separated from the triphosphates. If the duplexes are on beads, the last is simplest, and can be done by simply washing.

Continued extension generates a full length product. Depending on the size of the Covaris-fragmented target genome fragment and were within that fragment the primer primes, the resulting product will be ca. 100 base pairs in length. These can then be delivered for sequencing, for example, at a 454 DNA sequencing facility. There, the sequences are polished (made blunt ended) sequenced.

### Use of the 3′-NH₂ Unit.

This unit is an irreversible terminator. Polymerases that are well known in the art accept it on an incoming nucleotide triphosphate. Once incorporated, further extension is blocked; this blockage cannot be undone, as it can with the 3′-ONH₂ unit. Once incorporated, the 3′-NH₂ unit serves as a handle to capture a downstream oligonucleotide.

One way to subsequently capture the 3N products involves reacting a 3′-functionality introduced by the terminating unit to introduce a primer. A 5′-aldehyde DNA carrying a downstream restriction site, primer binding site, hairpin, and sequencing primer with a fluorescent tag will be synthesized following the procedure shown in FIG. 4.5, and then be captured on to the 3′-ONH₂ units of the primers extended in Aim 8. These will be between 1 and 0.1% of the beads in each primer set, approximately

Then, capture oligonucleotides with a 5′-aldehyde group will be prepared. The 5′-homologated aldehydes are known as 2′-deoxyribonucleosides, and will be prepared by the procedure shown in FIG. 4.5. These will be converted to the phosphoramidites via standard procedures, and added in the final coupling step in the synthesis of oligonucleotides. Should the reactivity of the aldehyde prove to be problematic, we will replace the C═O unit with a C═CH₂ unit. This will be converted to the diol, protected as the diacetate, and incorporated into the synthesis. The aldehyde will be generated from the diol generated in the oligonucleotide deprotection step by periodate cleavage.

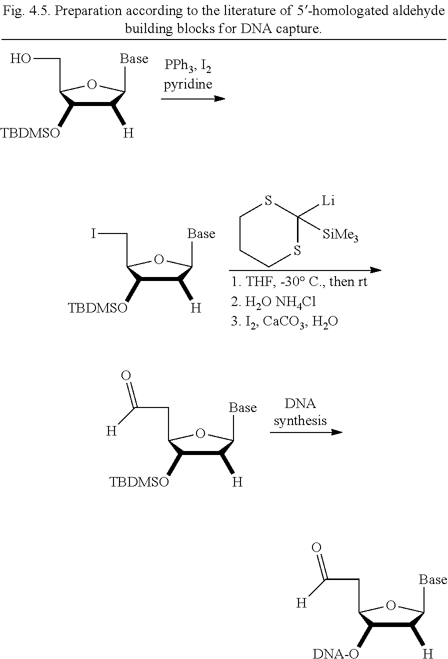

The resulting beads are sorted to separate those that discovered a SNP from those that did not If 0.1% of the sites are polymorphic in the high information segments of the human genome, the 1.5×10⁸ primers in each set will generated 1.5×10⁵ beads (150,000) that find a polymorphism. The Cytopedia sorter will require two minutes to sort each set of this size, to generate approximately 600,000 beads in 10 minutes of sorting. At the upper limit, if 1% of the sites are polymorphic in the high-information segments of the human genome, then 6 million beads will be delivered by the polymorphism-finding protocol. These will be sorted in ˜100 minutes.

The result of the second protocol is a set of beads holding the SNPs in the genome preceded by a 16 mer segment from the library that identifies the location of the SNP in the reference genome. This 17 mer (16+1) is flanked by primer binding sites and restriction sites. In the architecture shown in FIG. 4.4, the beads can be directly placed on the sequencing chips prepared by Intelligent Biosystems and directly sequenced.

Each of the prepared by Intelligent Biosystems chips has 40 million spots. We expect to get good sequence out of 30 million of these and 2 chips are run at a time. This permits the determination of ca. 60 million sequences from one run. The surface of the chip has 40 million holes that are about 5 um in diameter and 3 um deep. Thus, the 4.5 micron beads used in the split-and-pool synthesis fit directly in these. The entire surface is treated so that it may be crosslinked to the beads. The beads are spread across the chip and crosslinked; a 97% fill ratio is typically obtained. The chips are now ready to be sequenced. The sequencing is initiated using the hairpin attached to the captured aldehyde DNA as the sequencing primer (FIG. 4.4).

### Example 3

### Determining Heterozygosity in a Diploid Genome

When characterizing an individual diploid genome, it is useful to identify the differences that distinguish the genetic material that is maternally derived from the material that is paternally derived. The number of differences is on the order of the number of differences in the genomes separating two individuals in a species. Further, depending on the extent to which the parents are representative of the population as a whole, a SNP separating the maternal and paternal genomic endowments has a good to excellent chance of being a SNP that distinguishes the individual genome from the average genome of the population.

Guided by the teachings of the instant application, one of skill in the art will appreciate that many architectures may enable this identification. This Example presents one, for a genome without repeats, a genome that is formally both the reference and the target.

### Step 1.

Protocol 1. The physical DNA from the genome is fragmented using a Covaris instrument to yield double stranded fragments that are, preferably, 100+/−20 nucleotides in length. Fragments of this length require 100 genomes to have ca. 50% probability of having a primer for any given site. Therefore, 10,000 genome equivalents are preferably used.

Protocol 2. The fragments are then made blunt ended (“polished”), either by treatment with a 3′-single stranded exonuclease (a cut back protocol) or by treatment with DNA polymerase and 2′-deoxynucleoside triphosphates (a fill in protocol), or both. As is appreciated by those skilled in the art, not all fragments need to be polished for the products to have utility.

Protocol 3. The blunt ended duplex fragments are then separated into four portions, from which are created the G-extendable, A-extendable, T-extendable, and C-extendable primer sets. In this example, the ligation-cleavage protocol is used. For each portion, the blunt ended duplex fragments are ligated to a specific double stranded units that, upon restriction endonuclease treatment, generate one of the four primer sets with a free, extendable 3′-OH group. At the other end of the fragment, duplex attaches at the 5′-end of the strand a sequence a primer binding site (in the figure, this is represented as an 18 mer by N₁₈, although other primer binding site sequence lengths may be used, and the primer binding site may contain components of an artificially expanded genetic information system [Ben04]).

Protocol 4. The processed duplex fragments are then heated to separate the strands, preferably to over 80° C.

Protocol 5. The mixture is then slowly cooled to anneal the fragments that display complementarity. The cooling rate is preferably slower than 1 degree per minute.

The figure shows the details of this process for generating the G-extendable primer sets. Following melting and annealing (protocols 4 and 5), various kinds of duplexes can be formed. The simplest are duplexes that re-form the duplex between the two species that were processed in Protocol 2. If 10,000 genome equivalents were used, it is unlikely that these two would find each other amid other fragment strands. If they do, however, they are fully sequenced matched along their entire lengths, approximately 75% have a G-extendable OH group on only one 3′-end (and this extendable strand has the N₁₈ mer on the other end, and approximately 25% of them have a G-extendable OH group on both 3′-ends of the duplex, with both of the strands containing a 3′-extendable strands not having at their 5′-ends the N₁₈ primer binding site.

The remainder of the strands, upon the annealing in Protocol 5, will have found a strand as a duplex partner that was part of a different duplex generated in Protocol 1 and processed in Protocol 2. This partner will either come from the same parental lineage, or not. If it comes from the same parental lineage, then it will not contain a SNP. This means that the G-extendable-OH will he a 5′-underhang with a 3′-extension of four nucleotides, the first nucleotide of which will template the addition of a G to the 3′-OH group. The 5′-end of extendable strand now matched with a template that calls for extension with a G will, in 75% of the cases, carry the N₁₈ primer binding site; the remaining 25% will not.

If the partner comes from the other parental lineage and does not contain a SNP, then the duplexes arising upon annealing will be the same as the duplexes arising when the annealed duplex pairs material from the same parental lineage. If, however, the partner comes from the other parental lineage and does contain a SNP, then the fragment with the extendable 3′-OH end (if templated at all; it is possible that its partner places this 3′-OH nucleotide in a 3′-overhang) will be templated on a sequence that could not have been processed to create an extendable end.

The Figure shows some of the combinations of overhang and underhang that can be generated for primers that have found a SNP. The only primer-template pairs that can capture the SNP are those where the G-extendable primer is a 3′-underhang. Since this is a stochastic process and each site has (in the minimal case of 100 genome equivalents being processed) on the order of 100 opportunities to sample each SNP, a sufficient number of annealed pairs will have a 3′-underhang that can be extended with templating.

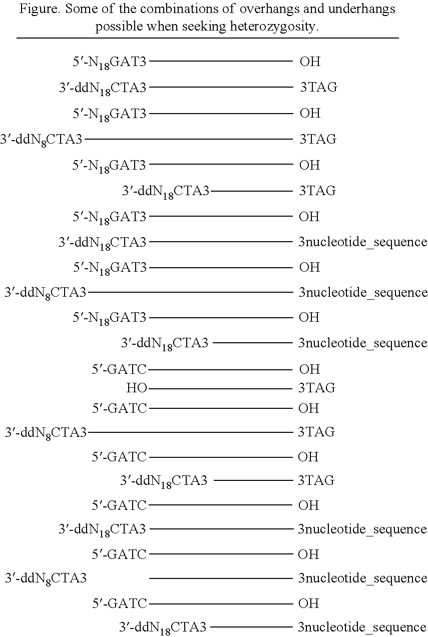

A variety of architectures can be employed to capture SNPs that have been found.

### Step 2. Use of an Incorporated Tag to Capture a Downstream Primer Binding Site

According to the method of the instant invention, when the G-extendable primers are extended with a G in step 2, they lead to a product that cannot be further processed, or that can be withdrawn from the pool that is delivered. When they are extended with “not G” (here, abbreviated as “3” to indicate “the other three nucleotides”).

In Step 2(a), the hollowing protocols are followed.

Protocol 6(a). The annealed template duplexes from Protocol 5 are incubated with DNA polymerase, preferably the Tabor-Richardson variant of Taq, 2′,3′-dideoxyguanosine triphosphate, and the 2′,3′-dideoxy-3′-aminonucleoside triphosphates of A, T, and C (the “3” nucleoside triphosphates). Both are terminators, adding just a single nucleotide to the 3′-extendable end.

Protocol 7(a). The products are then contacted with an oligonucleotide analog that will serve as a primer binding site, preferably 20 nucleotide units in length, having a preselected sequence chosen to not prime in the genome being analyzed, whose 5′-OH unit has been replaced by a HOCH₂—CH₂-unit. Following the formation of an imine, the mixture is treated with sodium cyanoborohydride, preferably with a final concentration of 10 mM, to reduce the imine to a secondary amine.

Protocol 8(a). The products are then PCR amplified (preferably 5 cycles), using a 5′-phosphorylated oligonucleotide complementary to the amine-linked downstream primer as the forward primer, and a 5′-phosphorylated N₁₈GAT oligonucleotide as the reverse primer.

Protocol 9(a). The PCR amplified products do not have a secondary amine linker, and are the only species in the mixture having ligatable 5′-phosphorylation and 3′-OH units. These are therefore delivered directly to the 454 sequencing protocol.

### Example 4

### Removal of Repeats

Picking just one repeat at random with no particular significance, the 100 mer TGTGGGAGTCTAAGTC TCTTTGTAGGTCACTCAGGACTTGCTTTATGAATCTGGGTGCTCCTGTATTGGGTGCAT ATATAT TTAGGATAGTTAGCTCTTC occurs 22 times in the in silico genome, in the repeats, far above that expected by any stochastic model. In contrast, a bioinformatics survey of the in silico human genome shows that while essentially all of the 4¹⁴ (≈268,435,546) possible 14 mers are found at least once in the human genome (the complement is not counted a second time in this analysis), about 10% of these 14 mers are found exactly once in the genome. This is consistent with the notion that a 14 gram string in general specifies a unique site for a segment of DNA in the non-repeating fraction of the genome.

To remove this repeat, we prepare sequences that terminate at its various points, and its complement. These are used to template the mixtures of primers, using a reversible terminator to

[symbol]

What is claimed is:

1. A process for generating a collection of oligonucleotides enriched in individual oligonucleotides, each of said individual oligonucleotide binds to a complementary sequence within a target DNA molecule wherein said sequence has a nucleotide replacement at a queried site distinguishing it from an analogous sequence within a reference DNA molecule, wherein said process comprises (i) providing of four sets of primers, called “T-extendable”, “A-extendable”, “C-extendable”, and “G-extendable”, wherein each set, when templated on the reference DNA sequence, is extended (respectively) using a polymerase by thymidine, adenosine, cytidine, or guanidine, (ii) contacting each set separately with target DNA under conditions where the primer can bind to a complementary sequence within the target DNA to form a duplex, and (iii) incubating said duplex with a polymerase to form extended products, wherein the extended products that are formed from T-extendable primers are different if they are extended by T than they are if they are extended by another nucleotide, the extended products that are formed from A-extendable primers are different if they are extended by A than they are if they are extended by another nucleotide, the extended products that are formed from C-extendable primers are different if they are extended by C than they are if they are extended by another nucleotide, and the extended products that are formed from G-extendable primers are different if they are extended by G than they are if they are extended by another nucleotide, and wherein said differences are used to enrich said collection.

2. The process of claim 1, wherein said differences are in the nature of a moiety appended to the 3′-carbon of the 3′-terminal nucleotide.

3. The nucleotide replacements and the flanking sequences wherein variation is found by the process of claim 1.
